(19) United States
(12) Patent Application Publication
(10) Pub. No.: US 2005/0149153 A1
(43) Pub. Date: Jul. 7, 2005

(54) Body temperature adjuster

(76) Inventor: Kazuo Nakase, Tokyo (JP)

Correspondence Address: Richard P. Berg, Esq.;c/o LADAS & PARRY, Suite 2100, 5670 Wilshire Boulevard, Los Angeles, CA, 90036-5679, US

(21) Appl. No.: 10/845,336

(22) Filed: May 12, 2004

(30) Foreign Application Priority Data
Jan. 7, 2004 (JP) 2004-1760

(51) Int. Cl. A61F 7/00

(52) U.S. Cl. 607/108; 607/112

(57) ABSTRACT

To provide a body temperature adjuster which is easy to wear, comfortable to wear, and provides safe and effective adjustment of the body temperature. A length adjustable band member, the band member including a plurality of first storages and a plurality of second storages for storing temperature-adjusting members for adjusting the body temperature are provided, and when the wearer puts on the band member, the first storages are disposed at the positions contacting the armpits where axillary arteries run through, and the second storages are disposed at the positions contacting both sides of the neck where common carotid arteries run through.

### BACKGROUND OF THE INVENTION

1. Field of the Invention

The present invention relates to a body temperature adjuster for controlling the body temperature using a cold insulator or a thermal insulator when the body temperature rises or drops due to common cold or frostbite.

2. Description of the Related Art

In the related art, when one has got Heat Disorder or febrile diseases, a cold insulator, a cold storage material or a cooling material is placed on his/her forehead to allow his/her body temperature to drop to a normal range. As a cooling device as those described above, a device for fixing a cover member (bag) for storing the cold insulator, on the forehead with a band is disclosed in JP-A-7-246215.

A device provided with adhesive means in a cover member for storing a cold insulator and adapted to adhere the cover member for storing a cold insulator to the forehead is also known.

In JP-A-2000-060891, a device intended to cool the armpits is disclosed. The device disclosed in JP-A-2000-060891 includes Scotch Magic™ Tapes on cold insulator bodies, and the cold insulator bodies are fixed to the armpits by putting a rubber belt which is detachably attachable to Scotch Magic™ tape to the back of the body in a cross-multiplying manner.

However, the device in JP-A-7-246215 is just for cooling the forehead, and thus cooling is effected only locally, which is not medically effective for bringing the body temperature down to a normal range. Fever generated in ecological tissue of the body is adapted to be transferred by the ecological tissue and circulating blood flow. The thermal conductivity of the ecological tissue is 0.001 cal/(s·cm²·° C.) for muscle and 0.0005 cal/(s·cm²·° C.) for fat without blood flow. In contrast, the thermal conductivity in the case where a calorie is transferred by blood flowing through blood vessels (100 μm in diameter), which is thicker than blood capillary (about 10 μm in diameter), at a flow rate of 1 ml/s is about 0.24 cal/(s·cm²·° C.). Therefore, even though the forehead is cooled, the body temperature cannot be brought down effectively, since blood vessels with a high flow rate of blood is not running under the forehead.

As devices other than that disclosed in JP-A-7-246215, a device for cooling the back part of the neck or the occipital region is used as a cooling device. However, these devices are also ineffective for bringing the body temperature down. On the contrary, cooling the back of the neck or the occipital region of newborn infants or babies causes sympathetic tone, which is dangerous because it may trigger ataxia or cerebral malfunction.

A device having adhesive means provided on the cover member for storing the cold insulator has a problem in that the cover member tends to come of f when rolling over in bed or during daily actions. In particular, it is disadvantageous in that when it is used for measures against Heat Disorder in the heat of summer, adhesive agent may loose its adhesive property (stickiness) due to perspiration and come off.

In addition, although the device disclosed in JP-A-2000-060891 is configured to cool the armpits, it is not configured to cool the head (brain, in particular) which is considered to be one of the most important parts of the body when suffering from Heat Disorder. Therefore, effective adjustment of the body temperature cannot be achieved. In addition, it is disadvantageous in that it cannot be used to fit wide range of physical constitution from children to adults, since adjustment is only made by elasticity of the rubber belt. Granting that it can be worn, it cannot be fixed securely since elasticity is too weak or it causes discomfort since elasticity is too strong.

### SUMMARY OF THE INVENTION

In view of such circumstances, it is an object of the present invention to provide an easy to wear, comfortable to wear, safe, and effective body temperature adjuster.

The above-described object is achieved by the provision of a length adjustable band member, a plurality of first storages and a plurality of second storages for storing temperature-adjusting members for adjusting the body temperature fixed in the band member, so that when the wearer puts on the band member, the first storages are disposed at the positions contacting the armpits where axillary arteries run through, and the second storages are disposed at the positions contacting both sides of the neck where common carotid arteries run through.

The above-described object is effectively achieved by configuring the first storages and the second storages in such a manner that the temperature-adjusting members can be detachably stored.

The above-described object is effectively achieved by the first storages and the second storages configured to be fine-adjustable in position on the band member depending on the wearer's physical constitution.

The above-described object is effectively achieved by the band member including a first band member for storing the first storages and a second band member for storing the second storages.

The above-described object is effectively achieved by the band member having one or more third storages for storing the temperature-adjusting member, and the third storage or storages being disposed at the position contacting the central portion of the back of the body when the wearer puts the band member on the body.

The above-described object is effectively achieved by the third storages configured so that the temperature-adjusting members can be detachably stored.

The above-described object is effectively achieved by the third storage or storages configured to be fine-adjustable in position on the band member depending on the wearer's physical constitution.

The above-described object is effectively achieved by the band member including a first band member having the first storages and the third storage or storages and a second band member having the second storages.

The above-described object is effectively achieved by the temperature-adjusting members being cold insulators having a cold reserving feature, or thermal insulators having a heat-reserving feature.

The above-described object is effectively achieved by one or more temperature sensors for measuring the wearer's body temperature, a temperature controller for adjusting heating or cooling of the temperature-adjusting members based on the value detected by the temperature sensor, and a power source for supplying an electric current to the temperature-adjusting members, and the temperature controller adjusting the temperature of the temperature-adjusting members by controlling the electric current supplied from the power source.

The above-described object is effectively achieved by the temperature controller including a ROM containing a standard body temperature in a table format, and a CPU for comparing the value detected by the temperature sensor with the standard body temperature whereby controlling the electric current to be supplied to the temperature-adjusting members.

The above-described object is effectively achieved by the temperature-adjusting member including a Peltier element to be controlled in temperature by the temperature controller and a heat conductive member encapsulating the Peltier element.

The body temperature adjuster according to the invention includes a length-adjustable band member, a plurality of first storages and a plurality of second storages provided on the band member for storing the temperature-adjusting members for adjusting the body temperature, and configured in such a manner that when the wearer puts the band member on the body, the first storages are disposed at the positions contacting the armpits where axillary arteries run through, and the second storages are disposed at the positions contacting both sides of the neck where common carotid arteries run through. Accordingly, the portions where circulating blood flow (thick vessel) having significant body temperature adjusting capability passes through can be simultaneously cooled or heated. As a consequence, an effective temperature adjustment is achieved, and hence the body temperature adjuster of the invention is effective for bringing down fever caused by febrile diseases, preventing Heat Disorder or chilling, or protecting the body from the cold.

In addition to the first and second storages, by the provision of the third storage or storages that come into contact with the portion near the aorta running through the central portion of the back of the body when the wearer puts the band member on the body, further effective adjustment of the body temperature is achieved.

The first and second storages for storing the temperature-adjusting members are fixed with the length adjustable band member. Accordingly, the body temperature-adjusting members are ensured to be fixed in position, and they are not displaced even during sleep, daily actions, or even during hard exercise or outdoor activities.

The first and second storages are configured in such a manner that the temperature-adjusting members are detachably stored. Accordingly, the band member can be washed after use. In addition, since the storages are of a shape corresponding to commercially available cold insulators and thermal insulators, even when the temperature-adjusting feature of the temperature-adjusting member has stopped, it can be used by storing the commercially available cold insulators or thermal insulators as a temperature-adjusting member.

The positions of the first storages, the second storages, and the third storages on the band member may be fine-adjusted depending on the wearer's physical constitution. Accordingly, it is ensured that the storages are disposed at the positions contacting the parts of the body to be adjusted in temperature even though the wearer is a small infant or an adult of large build.

Since the temperature-adjusting members are stored in the storages and do not come into direct contact with the body, the temperature of the surface of the portion which comes into contact with the body is prevented from being too hot or too cold. Accordingly, heat injury or frost injury of the body due to the temperature-adjusting member is prevented, so that a safe usage of the temperature adjuster is ensured.

The band member includes the first band having the first storages, and the second band having the second storages. Accordingly, the body temperature adjuster can easily be put on the body. In addition, since it comfortably fits the body, the wearer can go about his/her daily activities comfortably, while wearing the body temperature adjuster. As a consequence, newborn infants or babies who often run a fever can wear it comfortably without unwillingness.

The temperature-adjusting member includes a Peltier element, a conductive member encapsulating the Peltier element, one or more temperature sensors for measuring the wearer's body temperature, a temperature controller for controlling heating and cooling of the temperature-adjusting members based on the value detected by the temperature sensor, and a power source for supplying an electric current to the temperature-adjusting members, and the temperature controller is adapted to adjust the temperature of the temperature-adjusting members by controlling the electric current supplied from the power source. Accordingly, fine adjustment of heating and cooling of the body temperature adjuster can be performed utilizing the characteristics of Peltier effect that the heat moves in the direction of current flow and hence heat absorbing portion and heat generating portion are formed. As a consequence, the wearer's body temperature can easily be fine adjusted.

Furthermore, since the temperature controller includes the ROM containing the standard body temperature in a table format and the CPU for comparing the value detected by the temperature sensor with the standard body temperature, whereby controlling the current to be supplied to the temperature-adjusting members, the temperature of the temperature-adjusting members can be automatically adjusted, so that the wearer's body temperature is controlled to a standard body temperature which is preset as desired. Consequently, for example, when the body temperature adjuster is put on the wearer who is sleeping while suffering from fever, the body temperature adjuster can cool the armpits and both sides of the neck until the wearer's body temperature is brought down to the standard body temperature, and then stops the cooling function automatically, when the wearer's body temperature has reached the standard body temperature. Accordingly, the body temperature adjuster of the invention can be used for babies or newborn infants with security.

### BRIEF DESCRIPTION OF THE DRAWINGS

FIG. 1 is a perspective view of a body temperature-adjusting member according to a first embodiment of the present invention;

FIG. 2 is a side view of a principal portion of the body temperature adjuster;

FIG. 3 is a front view of the body temperature adjuster being worn on the body;

FIG. 4 is a back view of the body temperature adjuster being worn on the body;

FIG. 5 is a perspective view of the body temperature adjuster according to a second embodiment of the invention;

FIG. 6 is a front view of the body temperature adjuster being worn on the body;

FIG. 7 is a back view of the body temperature adjuster being worn on the body;

FIG. 8 is a perspective view of a body temperature adjuster according to a third embodiment of the invention;

FIG. 9 is a side view of a principal portion of the body temperature adjuster;

FIG. 10 is a back view of the body temperature adjuster being worn on the body;

FIG. 11 is a block diagram of a body temperature adjuster according to a fourth embodiment of the invention; and

FIG. 12 is a back view of a body temperature adjuster in the related art being worn on the body.

### DESCRIPTION OF THE PREFERRED EMBODIMENTS

Referring now to the drawings, embodiments of the present invention will be described.

[First Embodiment]

FIG. 1 to FIG. 4 show a body temperature adjuster according to a first embodiment of the invention. In FIG. 1, a body temperature adjuster 1 includes a first band member 2A having elasticity, a second band member 2B also having elasticity and being shorter than the first band member 2A, a connecting member 3 for connecting the first band member 2A and the second band member 2B, a pair of first storages 4A, 4A disposed on the first band member 2A so as to contact the armpits where axillary arteries run through when being worn, a pair of second storages 4B, 4B disposed on the second band member 2B so as to contact both sides of the neck where common carotid arteries run through when being worn, a first joining device 5A disposed at both ends of the first band member 2A for joining both ends of the first band member 2A, and a second joining device 5B disposed at both ends of the second band member 2B for joining both ends of the second band member 2B.

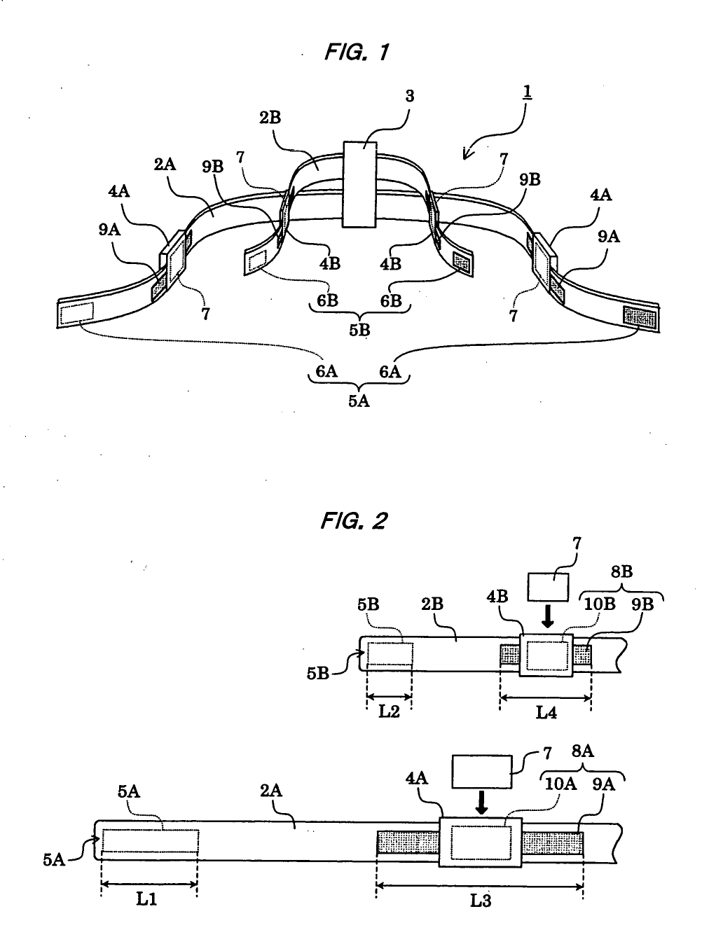

FIG. 2 shows a principal portion of the body temperature adjuster 1. The first joining device 5A includes a pair of hook-and-loop fasteners 6A, 6A disposed one on each surface at both ends for joining both ends of the first band member 2A when putting the body temperature adjuster 1 on the body. One or both of the hook-and-loop fasteners 6A, 6A have a predetermined length L1, so that the size or the tightening force can be adjusted when worn on the body. The second joining device 5B also includes a pair of hook-and-loop fasteners 6B, 6B disposed one on each surface at both ends for joining both ends of the second band member 2B. One or both of the hook-and-loop fasteners 6B, 6B have a predetermined length L2 (L2<L1) so that the size or the tightening force can be adjusted when worn on the body.

Since the second joining device 5B is a portion for joining the second band member 2B which is to be attached around the neck, in view of safety of the wearers such as babies or newborn infants, the joining force is adjusted so that the joint between the hook-and-loop fasteners 6B, 6B is disconnected immediately when the second band member 2B is caught by something and a force to tighten the neck is exerted.

The first storages 4A, 4A and the second storages 4B, 4B include temperature-adjusting members 7 respectively stored therein. These temperature-adjusting members 7 are commercially available cold insulators or thermal insulators. Therefore, when the wearer puts the body temperature adjuster 1 on the body, the wearer can obtain the cooling effect by storing the cold insulators in the respective storages 4, and in contrast, the wearer can obtain the heating effect by storing the thermal insulators therein.

A fixing device 8A for fixing between the first storages 4A and the first band member 2A also includes, as in the case of the joining device 5, hook-and-loop fasteners 9A disposed on the first band member 2A and a hook-and-loop fasteners 10A disposed on the back sides of the first storages 4A. The fasteners 9A have a predetermined length L3 so that the positions of the first storages 4A can be adjusted so as to contact the armpits of the wearers having different chest measurements. A fixing device 8B for fixing between the second storages 4B and the second band member 2B also includes hook-and-loop fasteners 9B disposed on the second band member 2B and hook-and-loop fasteners 10B disposed on the back sides of the second storages 4B. The fasteners 9B have a predetermined length L4 so that the positions of the second storages 4B can be adjusted so as to contact the portion near both sides of the neck where common carotid arteries run through when the wearers have different meansurements of the neck.

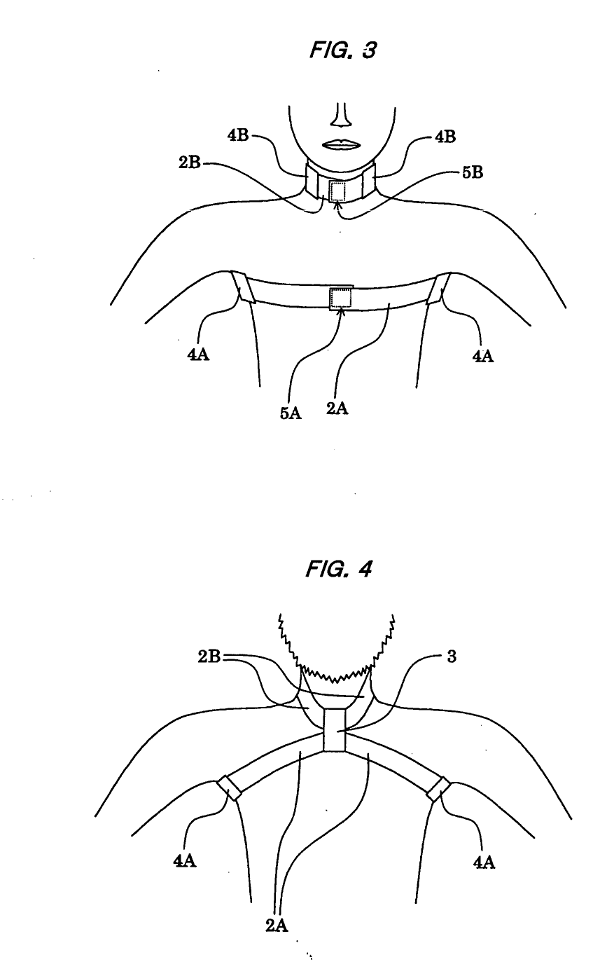

Subsequently, referring to FIG. 3 and FIG. 4, the operation of the body temperature adjuster 1 according to the present embodiment will be described.

FIG. 3 is a front view showing the body temperature adjuster 1 being worn on the body. FIG. 4 is a back view showing the body temperature adjuster 1 worn on the body. The temperature-adjusting members 7 adjusted to a proper temperature in advance are stored in storages 4 depending on the intended purposes, such as heating or cooling.

Subsequently, both ends of the first band member 2A is brought around from the back side passing under both arms to the chest, while adjusting the first storages 4A, 4A so as to be fixed at the positions contacting left and right armpits, and both ends of the first band member 2A are joined by means of the first joining device 5A.

Then both ends of the second band 2B are brought around from the back side passing the left and right sides of the neck to the front of the neck, while adjusting the second storages 4B, 4B so as to be fixed at the positions contacting the portion near common carotid arteries running through the left and right sides of the neck, and both ends of the second band member 2B are joined by the second joining device 5B.

As described above, according to the present embodiment, the first storages 4A, 4A having the temperature-adjusting members 7 stored therein are fixed to the positions contacting the armpits, and the second storages 4B, 4B are fixed to the positions contacting common carotid arteries running through both sides of the neck. Accordingly, the axillary arteries at the armpits and common carotid arteries at the neck where circulating blood flow having significant body temperature adjusting capability passes through can be cooled or heated simultaneously. As a consequence, in terms of the cooling effect, the body temperature can be effectively brought down when having a fever and hence is effective for prevention of diseases such as Heat Disorder. In terms of the heating effect, it is effective for protection from the cold or improvement of cold constitution.

The joining device 5 can adjust the size or the tightening force when being worn, and the fixing device 8 can adjust the fixing positions so that the storages 4 are disposed at the suitable positions when being worn. Accordingly, the body temperature adjuster of the present embodiment can be used by adults and children having different physical constitutions.

Furthermore, the band member 2, the storages 4, the temperature-adjusting members 7 can be detached respectively. Therefore, when they become dirty, these members can be detached for washing. When the temperature adjusting function of the temperature-adjusting members 7 has stopped, the commercially available cold insulators or thermal insulators can be stored in the storages 4 for reuse.

Although the first band member 2A and the second band member 2B are connected by the connecting member 3 in the present embodiment, it is also possible to use the first band member 2A and the second band member 2B separately without providing the connecting member 3. The joining device 5 is not limited to the hook-and-loop fastener 6 and may be buttons or strings.

The body temperature adjuster 1 of the present embodiment is configured in such a manner that the band member 2 is elastic and size-adjustable so that those having different physical constitutions can use. However, it may be provided in several preset sizes according to the intended users such as newborn infants, babies, men, and women.

[Second Embodiment]

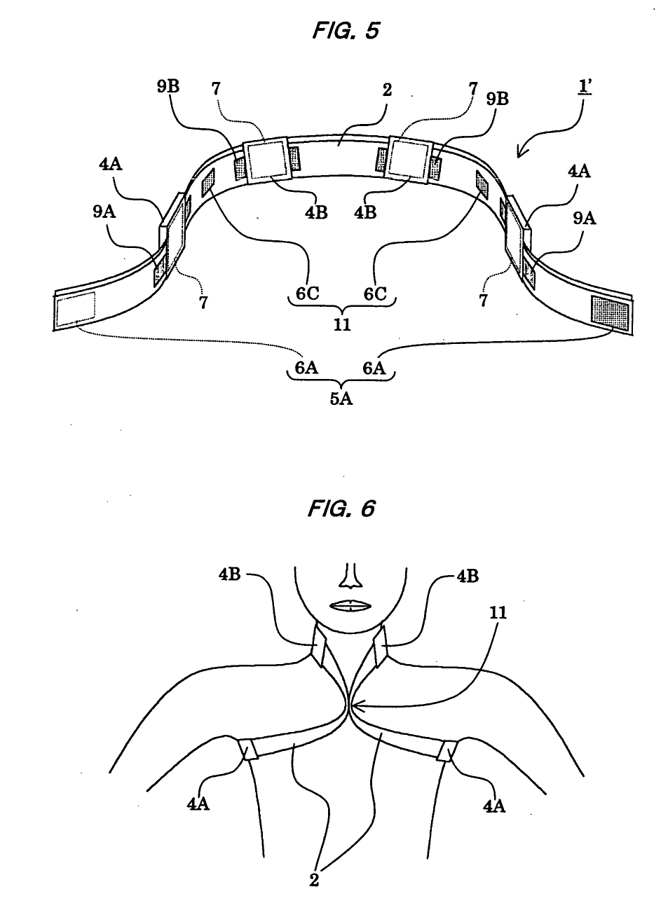

FIG. 5 to FIG. 7 show the body temperature adjuster according to a second embodiment of the present invention. The identical parts as in the above-described member are represented by the identical reference numerals and description will be omitted.

As shown in the perspective view in FIG. 5, a body temperature adjuster 1′ includes a band member 2 having elasticity, a pair of first storages 4A, 4A disposed on the band member 2 so as to contact the armpits where axillary arteries run through when being worn, a pair of second storages 4B, 4B disposed at the positions closer to the center of the band member 2 with respect to the first storages 4A, 4A so as to contact both sides of the neck where common carotid arteries run through, the joining device 5A disposed on both ends of the band member 2 for joining both ends of the band member 2, and a coupling device 11 disposed between the first storages 4A, 4A and the second storages 4B, 4B.

The coupling device 11 includes a pair of hook-and-loop fasteners 6C, 6C on the back side of the band member 2. The hook-and-loop fasteners 6C, 6C are configured to join the band member 2 while adjusting the positions of the second storages 4B, 4B so as to contact the common carotid arteries running through both sides of the neck when the wearer puts the body temperature adjuster 1′ on the body. One or both of the hook-and-loop fasteners 6C, 6C have a predetermined length so that the size or the tightening force can be adjusted when putting on the body.

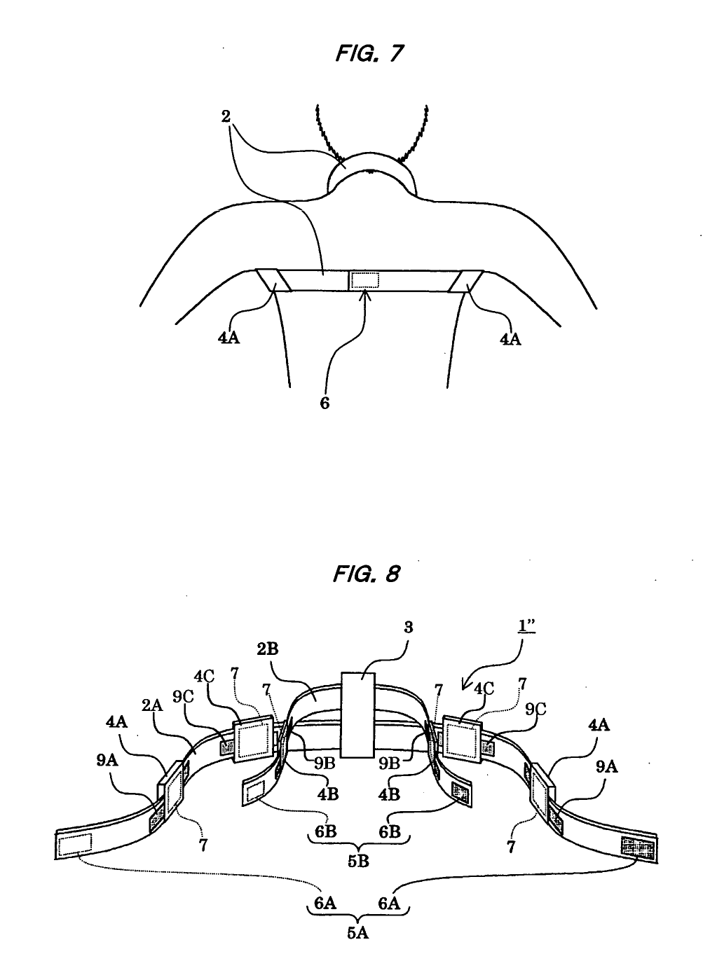

Referring now to FIG. 6 and FIG. 7, the operation of the body temperature adjustors 1′ according to the present embodiment will be described.

FIG. 6 is a front view of the body temperature adjuster 1′ being worn on the body, and FIG. 7 is a back view of the body temperature adjuster 1′ being worn on the body. First, the temperature-adjusting members 7 adjusted to a proper temperature in advance are stored in the storages 4 depending on the intended purposes, such as heating or cooling.

Then, the center of the back surface of the band member 2 is contacted the center of the back side of the neck, and from this state, both ends of the band member 2 are brought around to the front of the body by passing both sides of the neck. In this procedure, the positions of the second storages 4B, 4B are adjusted so as to contact the common carotid arteries running through both sides of the neck, and then the band member 2 is joined by means of the coupling device 11. Subsequently, from this state, both ends are brought to the back side through the left and right armpits while adjusting the position of the first storages 4A, 4A so as to contact the armpits, and both ends of the band member 2 are joined by the joining device 5A.

As described above, the pair of first storages 4A, 4A and the pair of second storages 4B, 4B are provided on a single band member 2 in the present embodiment. Therefore, the body temperature adjuster 1′ in the present embodiment achieves not only the equivalent effects as in the first embodiment, but also cost reduction since only one band member 2 is provided.

Since the body temperature adjuster 1′ of the present embodiments is configured not to tighten the neck, it can be used safely for babies or newborn infants.

Although the loop-and-hook fasteners 6 are used as the coupling deice 11 in the present embodiment, buttons or strings may be used as long as they provide a coupling force sufficient for preventing from being detached by rolling over in bed or some extent of movement.

[Third Embodiment]

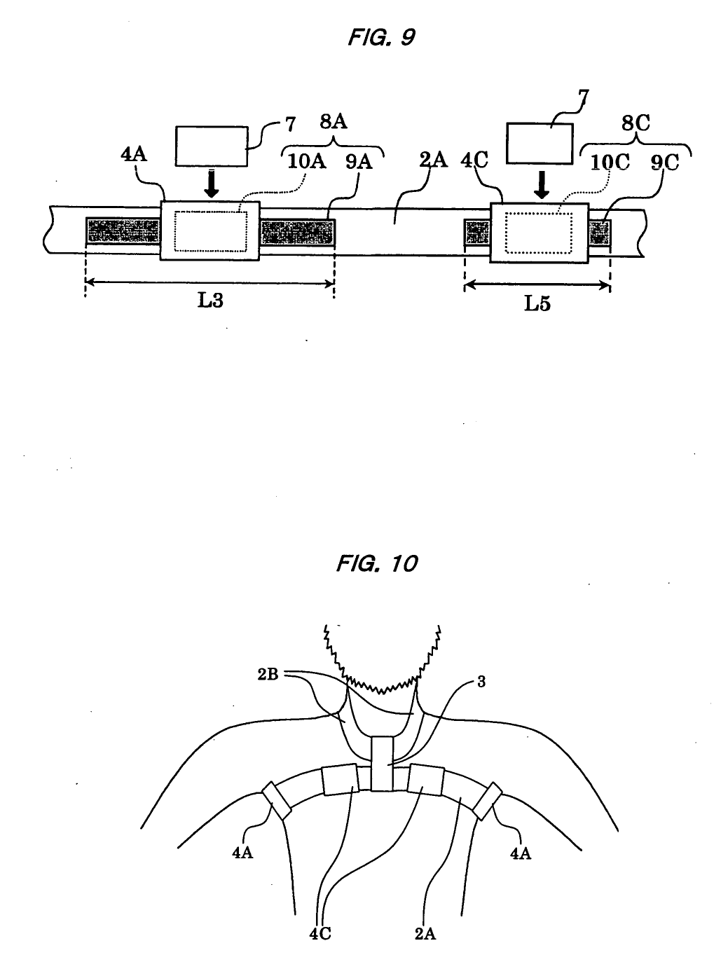

FIG. 8 and FIG. 10 show a body temperature adjuster according to the third embodiment of the invention. The identical parts as in the above-described embodiments are represented by the identical reference numerals and description will be omitted.

As shown in a perspective view in FIG. 8, a body temperature adjuster 1″ includes a pair of third storages 4C, 4C disposed on the first band member so as to contact the central portion of the back of the body where the aorta runs through when being worn in addition to the first storages 4A, 4A and the second storages 4B, 4B. The third storages 4C, 4C include the temperature-adjusting members 7 stored therein like the first storages 4A, 4A and the second storages 4B, 4B.

FIG. 9 shows a principal portion of the temperature-adjusting member 1″. A fixing device 8C for fixing the third storages 4C and the first band member 2A includes, as in the case of the fixing device 8A, hook-and-loop fasteners 9C disposed on the first band member 2A, and hook-and-loop fasteners 10C disposed on the back side of the third storage 4C. The fastener 9C has a predetermined length L5 so that the position of the third storages 4C can be adjusted contact the center portion of the back of the wearer when the wearer has a different chest measurement.

Referring now to FIG. 10, the operation of the body temperature adjuster 1″ according to the present embodiment will be described.

FIG. 10 is a back view of the body temperature adjuster 1″ worn on the body. First, the temperature-adjusting members 7 adjusted to a proper temperature in advance depending on the intended purposes, such as heating or cooling, are stored in the storages 4.

Then, both ends of the first band member 2A are brought around from the back side through both armpits to the chest, while adjusting the first storages 4A, 4A so as to be fixed at the positions contacting left and right armpits, and the third storages 4C, 4C so as to be fixed at the positions contacting the central portion of the back of the body, and both ends of the first band member 2A are joined by means of the first joining device 5A.

Then, both ends of the second band 2B are brought around from the back side passing the left and right sides of the neck to the front of the neck, while adjusting the second storages 4B, 4B so as to be fixed at the positions contacting the common carotid arteries running through the left and right sides of the neck, and both ends of the second band member 2B are joined by means of the second joining device 5B.

As described above, according to the present embodiment, the third storages that contact the central portion of the back of the body when being worn in addition to the first storages 4A, 4A and the second storages 4B, 4B are provided. Accordingly, the body temperature adjuster 1″ in the present embodiment achieves not only the equivalent effects as in the first embodiment, but also cooling or heating of the aorta at the central portion of the back of the body where circulating blood flow having significant body temperature adjusting capability passes through. As a consequence, further effective adjustment of the body temperature is achieved.

[Fourth Embodiment]

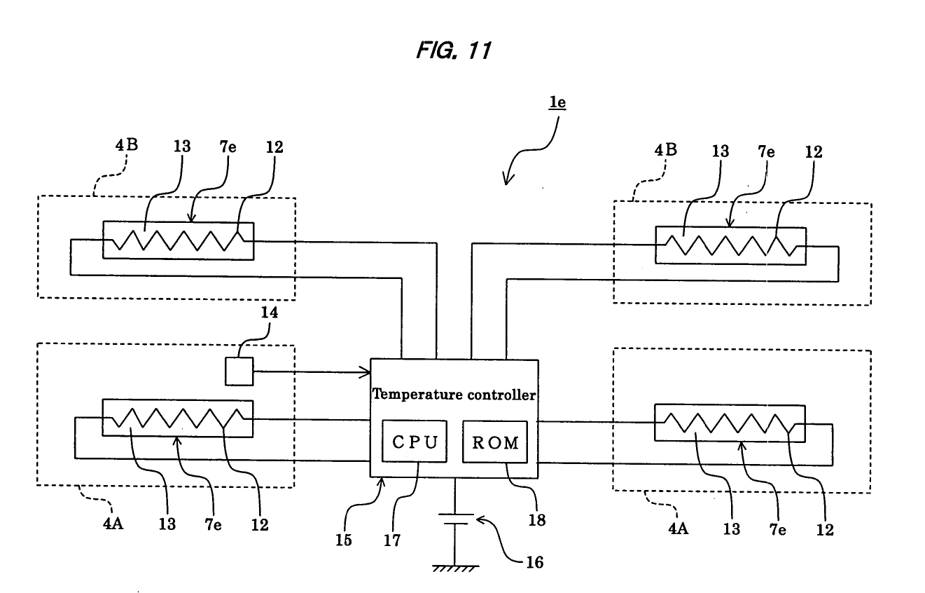

FIG. 11 shows a body temperature adjuster according to the fourth embodiment of the invention. In FIG. 11, the identical parts as in the above-described embodiments are represented by the identical reference numerals and description will be omitted.

The temperature-adjusting members 7 are commercially available cold insulators or thermal insulators in the above-described embodiments. However, in the present embodiment, the temperature-adjusting member is provided with a thermo-module structure, which can adjust the temperature of the temperature-adjusting member by adjusting an electric current to be supplied. The appearance of the body temperature adjuster according to the present embodiment is the same as the body temperature adjuster 1 according to the first embodiment shown in FIG. 1.

In FIG. 11, the reference numeral 4A designates the first storage shown in the above-described embodiment, and the reference numeral 4B designates the second storage of the same. The first storages 4A and the second storages 4B each include a temperature adjustor 7e including a Peltier element 12 that can adjust the temperature by the amount of electric current or the direction of current flow, and a heat conductive body 13 (gel, or the like) being superior in heat conductive property and encapsulating the Peltier element 12.

A temperature adjuster le includes a temperature sensor 14 for measuring the temperature of the wearer, a temperature controller 15 for adjusting heating or cooling of the temperature-adjusting members 7e based on the value detected by the temperature sensor 14, and a power source 16 for supplying an electric current to the temperature-adjusting members 7e.

The temperature controller 15 includes a CPU 17 and a ROM 18. The temperature controller 15 adjusts the temperature of the temperature-adjusting members 7e by comparing the detected body temperature from the temperature sensor 14 with a table of the standard body temperature stored in the ROM 18 in advance, calculating it with the CPU 17, and controlling the current supplied from the power source 16 to the temperature-adjusting members 7e so that the wearer's body temperature is adjusted to the standard body temperature. The standard temperature, which differs from person to person, can be set by the wearer as desired with a standard body temperature setting button provided on the temperature controller 15. Alternatively, for example, the standard body temperature may be changed in accordance with the intended use, such as setting the standard temperature to a relatively higher value when it is used for cold protection or for a measure against cold constitution.

The Peltier element 12 of the temperature-adjusting member 7e may have a configuration including a plurality of pairs of elements including P-type (+) thermoelectric material and n-type (−) thermoelectric material, and having planar metal strips, with plate-shaped electrodes provided on both surfaces. The Peltier element 12 generates heat when an electric current flows from the p-type thermoelectric material to the n-type thermoelectric material, and on the contrary, cools when the electric current flows from the n-type thermoelectric material to the p-type thermoelectric material. The heat generated by the Peltier element 12 is transferred to the armpits or both sides of the neck via the heat conducting member 13 and the respective storages 4.

As described thus far, according to the present embodiment, the temperature-adjusting member 7e includes the Peltier element 12, the heat conductive member 13 encapsulating the Peltier element 12 therein, one or more temperature sensors 14 for measuring the wearer's body temperature, the temperature controller 15 for adjusting heating or cooling of the temperature-adjusting member 7e based on the value detected by the temperature sensor 14, and the power source 16 for supplying an electric current to the temperature-adjusting member 7e, and the temperature controller is configured to adjust the temperature of the temperature-adjusting member by controlling the current supplied from the power source. Accordingly, the same effects as the first embodiment are achieved, as a matter of course, and the body temperature can be fine-adjusted by utilizing the characteristics of Peltier effect that heat moves in the direction of current flow and hence heat absorbing portion and heat generating portion are formed.

The body temperature adjuster may be configured by a pipe disposed so as to contact the armpits and both sides of the neck, circulating water circulating in the pipe for transferring heat, a temperature-adjusting member 7e for heating or cooling the circulating water, and a pump for circulating the circulating water, so that the armpits and the both sides of the neck can be heated or cooled by the circulating water. Accordingly, since the body temperature adjuster can do with one temperature-adjusting member 7e at the minimum, cost can be reduced, and reduction of power consumption is achieved.

While the detailed embodiments of the present invention have been described, it is to be understood that the invention is not limited to the disclosed embodiments. On the contrary, various modifications may be made without departing the scope of the invention.

1. A body temperature adjuster comprising:
a length adjustable band member; and
a plurality of first storages and a plurality of second storages for storing temperature-adjusting member for adjusting the body temperature fixed the band member,
wherein when the wearer wears the band member, the first storages are disposed at the positions contacting the armpits where axillary arteries run through, and the second storages are disposed at the positions contacting both sides of the neck where common carotid arteries run through.

2. A body temperature adjuster according to claim 1, wherein the first storages and the second storages are configured in such a manner that the temperature-adjusting members can be detachably stored.

3. A body temperature adjuster according to claim 1, wherein the first storages and the second storages are configured to be fine-adjustable in position on the band member depending on the wearer's physical constitution.

4. A body temperature adjuster according to claim 1, wherein the band member comprises a first band member for storing the first storages and a second band member for storing the second storages.

5. A body temperature adjuster according to claim 1, wherein the band member comprises one or more third storages for storing the temperature-adjusting member, and the third storage or storages are disposed at the position contacting the central portion of the back of the body when the wearer puts the band member.

6. A body temperature adjuster according to claim 5, wherein the third storages are configured so that the temperature-adjusting members can be detachably stored.

7. A body temperature adjuster according to claim 6, wherein the third storage or storages are configured to be fine-adjustable in position on the band member depending on the wearer's physical constitution.

8. A body temperature adjuster according to claim 5, wherein the band member comprises a first band member having the first storages and the third storage or storages and the second band member having the second storages.

9. A body temperature adjuster according to claim 1, wherein the temperature-adjusting members are cold insulators having a cold reserving feature, or thermal insulators having a heat reserving feature.

10. A body temperature adjuster according to claim 1, comprising:
one or more temperature sensors for measuring the wearer's body temperature;
a temperature controller for adjusting heating or cooling of the temperature-adjusting members based on the value detected by the temperature sensor; and
a power source for supplying an electric current to the temperature-adjusting members,
wherein the temperature controller adjusts the temperature of the temperature-adjusting members by controlling the electric current supplied from the power source.

11. A body temperature adjuster according to claim 10, wherein the temperature controller comprises:
a ROM containing a standard body temperature in a table format; and
a CPU for comparing a value detected by the temperature sensor with the standard body temperature, whereby controlling a current to be supplied to the temperature-adjusting members.

12. A body temperature adjuster according to claim 10, wherein the temperature-adjusting member comprises:
one or a plurality of Peltier elements to be controlled in temperature by the temperature controller; and
a heat conductive member encapsulating the Peltier element.
